(19) United States
(12) Patent Application Publication
(10) Pub. No.: US 2025/0054607 A1
(43) Pub. Date: Feb. 13, 2025

(54) IMAGING SUPPORT APPARATUS, IMAGING SUPPORT METHOD, AND IMAGING SUPPORT PROGRAM

(71) Applicant: Canon Medical Systems Corporation, Otawara-shi (JP)

(72) Inventors: Shuki MARUYAMA, Nasushiobara (JP); Hidenori TAKESHIMA, Tokyo (JP)

(73) Assignee: Canon Medical Systems Corporation, Otawara-shi (JP)

(21) Appl. No.: 18/752,889

(22) Filed: Jun. 25, 2024

(30) Foreign Application Priority Data
Aug. 8, 2023 (JP) 2023-129251

(51) Int. Cl. G16H 30/40; G06V 10/24; G06V 10/74; G16H 30/20

(52) U.S. Cl. CPC G16H 30/40; G06V 10/242; G06V 10/761; G16H 30/20

(57) ABSTRACT

According to one embodiment, an imaging support apparatus includes processing circuitry. The processing circuitry is configured to acquire a first image and a second image older than the first image. The processing circuitry is configured to calculate a correction value for reproducing the second image. The processing circuitry is configured to determine reproducibility of the second image based on the correction value.

### CROSS-REFERENCE TO RELATED APPLICATIONS

This application is based upon and claims the benefit of priority from Japanese Patent Application No. 2023-129251, filed Aug. 8, 2023, the entire contents of which are incorporated herein by reference.

### FIELD

Embodiments described herein relate generally to an imaging support apparatus, an imaging support method, and an imaging support program.

### BACKGROUND

A typical diagnostic imaging test assumes a flow in which, after settings such as placing a subject on a gantry and fixing an imaging target site, an imaging range is set by selecting a protocol and then imaging is initiated. Herein, depending on the condition of the subject, appropriate settings suitable for the disease and case are not always possible. Thus, a cross section desired by a medical staff may not be produced.

Furthermore, if a follow-up observation for the same subject involves variations in setting by each operator such as a technologist, deterioration is assumed in quality and speed of image diagnosis. For this reason, imaging with a reproducible setting is required.

### BRIEF DESCRIPTION OF THE DRAWINGS

FIG. 1 is a block diagram showing an imaging support apparatus according to a present embodiment.

FIG. 2 is a flowchart showing an example of operation of the imaging support apparatus according to the present embodiment.

FIG. 3 is a diagram showing an example of a past image and an imaged image which requires correction.

FIG. 4 is a conceptual diagram showing an example of a transformation parameter according to the present embodiment.

FIG. 5 is a diagram showing a first application example of a trained model according to the present embodiment.

FIG. 6 is a diagram showing a second application example of the trained model according to the present embodiment.

FIG. 7 is a diagram showing a third application example of the trained model according to the present embodiment.

FIG. 8 is a diagram showing an example of a display screen relating to a correction value according to the present embodiment.

### DETAILED DESCRIPTION

In general, according to one embodiment, an imaging support apparatus includes processing circuitry. The processing circuitry is configured to acquire a first image and a second image older than the first image. The processing circuitry is configured to calculate a correction value for reproducing the second image. The processing circuitry is configured to determine reproducibility of the second image based on the correction value.

Hereinafter, an imaging support apparatus, an imaging support method, and an imaging support program according to the present embodiment will be described with reference to the drawings. In the following embodiments, elements assigned the same reference numeral perform the same operation, and redundant descriptions will be omitted as appropriate. Hereinafter, one embodiment will be described with reference to the accompanying drawings.

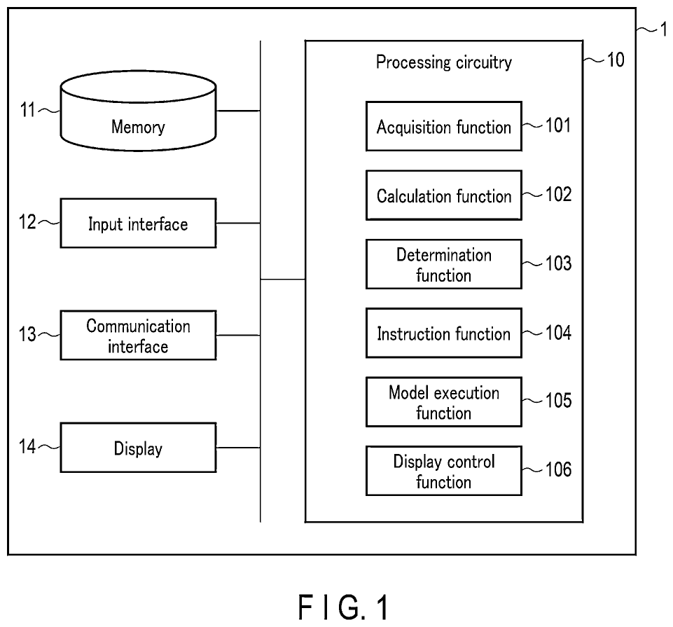

The imaging support apparatus according to the present embodiment will be described with reference to the block diagram of FIG. 1. The imaging support apparatus may be included in a console of a medical image diagnostic apparatus (not shown) or may be included in a computer independent of the medical image diagnostic apparatus and be communicably coupled to the image diagnostic apparatus.

An imaging support apparatus 1 according to the present embodiment includes a processing circuitry 10, a memory 11, an input interface 12, a communication interface 13, and a display 14.

The processing circuitry 10 includes an acquisition function 101, a calculation function 102, a determination function 103, an instruction function 104, a model execution function 105, and a display control function 106. The processing circuitry 10 includes a processor (not shown) as a hardware resource.

The acquisition function 101 acquires a first image and a second image older than the first image. The first image and the second image are medical images and are assumed to be, but are not limited to, a pair of images collected by the same medical image diagnostic apparatus, such as a pair of MR images collected by a magnetic resonance imaging (MRI) device, a pair of CT images collected by an X-ray computed tomography (CT) device, a pair of X-ray images collected by an X-ray diagnostic device, a pair of ultrasound images collected with an ultrasound diagnostic device, a pair of PET images collected with a positron emission tomography (PET) device, a pair of SPECT images collected with a single photon emission computed tomography (SPECT) device, etc. It suffices that the first image and the second image are acquired from, for example, a medical image diagnostic apparatus, a picture archiving and communication system (PACS) server, or various image processing servers.

For example, the first image may be a CT image, and the second image may be an MR image. That is, the first image and the second image may be a combination of medical images acquired by different medical image diagnostic apparatuses, such as MR images, CT images, ultrasound images, PET images, SPECT images, etc. The second image is not limited to medical images imaged by the same medical image diagnostic apparatus, and an image whose photographing date and time are close to those of the first image may be selected as the second image.

The calculation function 102 calculates a correction value for reproducing the second image. The correction value is, for example, the movement amount and the rotation angle of an imaging target site extracted in the first image. Furthermore, the correction value may be a parameter for extracting and reproducing the second image from the three-dimensional model.

The determination function 103 determines reproducibility of the second image based on a correction value. The reproducibility corresponds to a criterion for determining whether or not a setting for imaging based on a correction value relating to image alignment or image comparison of the first image with the second image is possible or not.

In a case where a setting based on the correction value is possible for a subject, the instruction function 104 generates an instruction for executing imaging or for correcting a setting. Alternatively, in a case where a setting based on the correction value is not possible for an imaging target site of the subject, the instruction function 104 generates a message indicating that a past image is unreproducible. Furthermore, the instruction function 104 performs a correction instruction of the setting for making an image close to a past image.

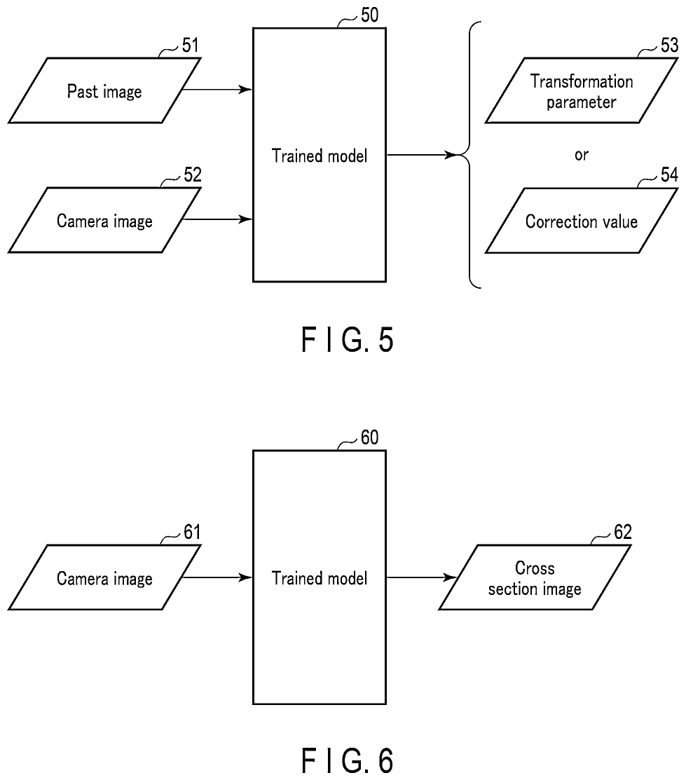

The model execution function 105 executes a trained model, thereby estimating a cross section image, a transformation parameter, or a correction value. The transformation parameter is a parameter for use in transforming an image in the present embodiment, and assumes parameters relating to the movement direction and rotation direction. However, the transformation parameter is not limited to them, and any parameter relating to image transformation, such as an enlargement rate or a reduction rate, may be adopted. Meanwhile, the trained model will be described later with reference to FIG. 5 to FIG. 7.

The display control function 106 causes the display 14, etc., to display an execution result of the trained model executed by the model execution function 105 (for example, a cross section image, a transformation parameter, and a correction value), a correction instruction by the instruction function 104, an unreproducible status of the past image, a correction instruction of the setting for making an image close to the past image, display of a difference, and so on.

The memory 11 stores a variety of medical data and acquisition conditions, trained models, etc., which will be described later. The memory 11 is a semiconductor memory element, such as a random access memory (RAM) and a flash memory, a hard disk drive (HDD), a solid state drive (SSD), an optical disk, etc. The memory 11 may also be a driver, etc., which reads and writes a variety of information from and to a portable storage medium such as a CD-ROM drive, a DVD drive, a flash memory, etc.

The input interface 12 includes a circuitry that receives various instructions and information input from a user. The input interface 12 includes a circuitry relating to a pointing device such as a mouse or an input device such as a keyboard, for example. The circuitry included in the input interface 12 is not limited to a circuitry relating to a physical operational component, such as a mouse or a keyboard. For example, the input interface 12 may include an electrical signal processing circuitry which receives an electrical signal corresponding to an input operation from an external input device provided separately from the imaging support apparatus 1 and outputs the received electrical signal to various circuitry.

The communication interface 13 executes data exchange with an external device by wired or wireless connections. A common communication means may be used as a communication means. Thus, a description of a communication method and an interface configuration will be omitted.

The display 14 displays a graphical user interface (GUI) for accepting various operations from a user. As the display, any display equipment may be discretionarily employed, including a cathode ray tube (CRT) display, a liquid crystal display, an organic EL display, an LED display, a plasma display, and a touch display which allows for touch input operations. However, the imaging support apparatus 1 may not include the display, and GUIs may be displayed on an external display or may be displayed via a projector, etc.

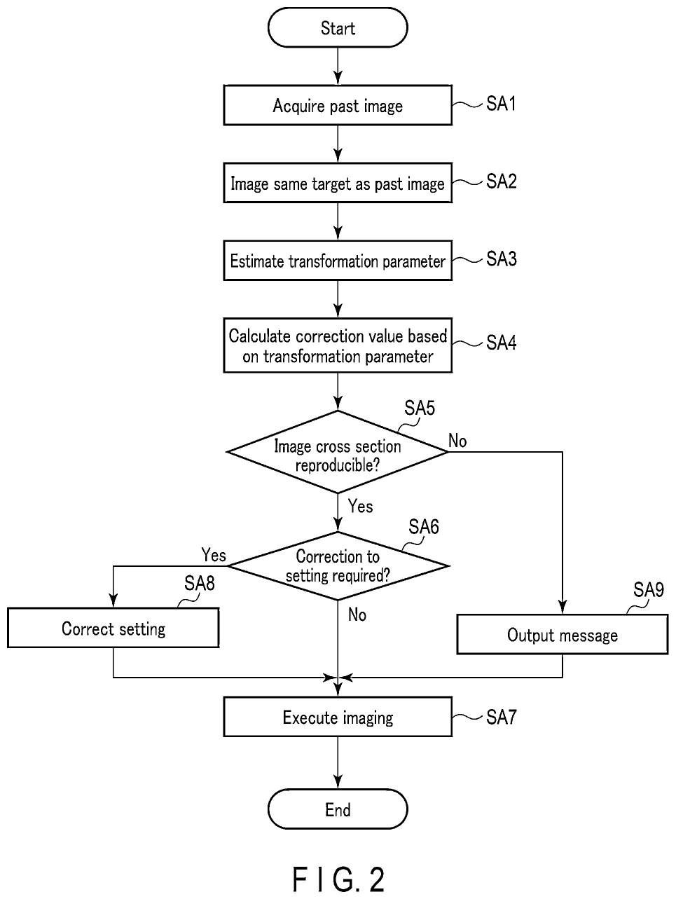

Next, an example of the operation of the imaging support apparatus 1 according to the present embodiment will be described with reference to the flowchart of FIG. 2.

In step SA1, the processing circuitry 10 acquires an imaged past image (the second image) through the acquisition function 101.

In step SA2, the processing circuitry 10 acquires an imaged image (the first image) relating to the same subject as that of the past image through the acquisition function 101. The imaged image acquired in step SA2 may be a positioning image or an imaged image collected by a main scan.

In step SA3, the processing circuitry 10 calculates, through the calculation function 102, a transformation parameter such that a similarity degree to the past image is maximized by transformation processing on the imaged image. Specifically, a transformation parameter in a case in which the imaged image is transformed linearly or nonlinearly may be estimated such that the imaged image becomes identical to the past image, for example. For example, in a case where a given point Xa in the imaged image is transformed into a point Xr in the past image, a transformation parameter is calculated based on an Expression of T (Xa)=Xr. Herein, T ( ) is a transformation parameter.

In step SA4, the processing circuitry 10 calculates, through the calculation function 102, a correction value based on the transformation parameter.

In step SA5, the processing circuitry 10 determines, through the determination function 103, whether or not a cross section is reproducible. Specifically, the processing circuitry 10 determines whether or not the correction value is equal to or smaller than a threshold value. The threshold value is assumed to be different depending on the imaging target site. For example, since a bending angle of the neck and a bending angle of the knee differ in a movable range, threshold values respectively corresponding to imaging target sites are set. Meanwhile, a comparison between a correction value and a threshold value assumes a comparison between an absolute value of the correction value and the threshold value in consideration of whether a transformation parameter is positive or negative. However, a positive threshold and a negative threshold may be provided to compare the correction value with these two thresholds. That is, in a case of the correction value being negative, the correction value may be compared with the negative value. In a case of the correction value being positive, the correction value may be compared with the positive threshold.

In a case of the correction value being equal to or smaller than the threshold, it is determined that the cross section of the past image is reproducible, and the processing proceeds to step SA6. On the other hand, in a case of the correction value being greater than the threshold value, correction cannot be made even with an adjustment to a setting. In this case, it is determined that the cross section of the past image is unreproducible and the processing proceeds to step SA7.

In step SA6, the processing circuitry 10 determines, through the determination function 103, whether or not correction of a setting is required. Specifically, in a case where the correction value is zero, it can be determined that no correction of a setting is required, and imaging can be executed as it is without changing the current setting. On the other hand, in a case where the correction value is greater than zero, it is determined that correction of a setting is required. Meanwhile, in a case where the correction value is not zero but is a value such that correction of a setting is not required, it may be determined that no correction is required, even if the correction value is greater than zero. In a case where correction of a setting is required, the processing proceeds to step SA8. In a case where no correction of a setting is required, the processing proceeds to step SA7.

In step SA7, since the cross section of the past image can be reproduced with a current setting, the processing circuitry 10 generates an imaging instruction for execution of imaging with the aforementioned setting through the instruction function 104, for example, so that the imaging is executed by the medical image diagnostic apparatus.

In step SA8, the processing circuitry 10 outputs an instruction for correction of a setting through the instruction function 104 and the display control function 106. In a case where the setting is corrected, the reproducibility of the cross section of the past image is improved, and the processing proceeds to step SA7 to execute imaging.

With respect to correction of a setting, the processing circuitry 10 presents a correction value to an operator by causing the display 14 to display the correction value through the display control function 106. Thereafter, correction may be made by an operator directly moving or bending a subject or the operator asking a subject to move or bend the subject's body. Alternatively, for example, the processing circuitry 10 may generate, through the instruction function 104, a control instruction for a controller configured to drive a device such as a bed of the medical image diagnostic apparatus. A setting may be automatically corrected by the bed of the medical image diagnostic apparatus being controlled based on the aforementioned instruction and the apparatus or the bed thus rotating and moving based on the correction value.

In step SA9, since a past image cannot be produced even by correcting the setting, the processing circuitry 10 generates, through the instruction function 104, a message indicating that the past image is unreproducible, and displays this message to a user through the display control function 106. Meanwhile, even in a case where a past image is unreproducible, in order to increase the reproducibility as close as possible to the past image, the processing circuitry 10 may generate, through the instruction function 104, a message including a correction instruction of the setting for making an image closest to the past image and a difference from the past image as corrected based on the aforementioned correction instruction and present the message to a user through the display control function 106. Thereafter, the processing proceeds to step SA7 and imaging is made executable.

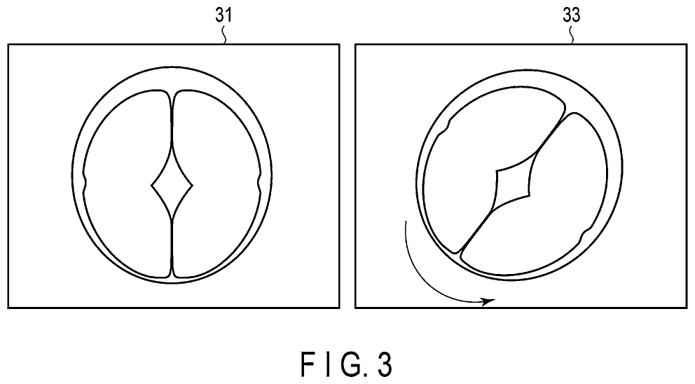

Next, an example of a past image and an imaged image which requires correction will be described with reference to FIG. 3.

FIG. 3 is a medical image with a target site set to the head, and shows a past image 31 and an imaged image 33 of the same patient. In the example of FIG. 3, the imaged image 33 is an image which is rotated clockwise in relation to the past image 31. Therefore, in order to align the past image 31 with the imaged image 33, the imaged image 33 needs to rotate in the direction of the arrow in FIG. 3, that is, counterclockwise. As a correction value, values such as the amount of movement and the amount of rotation, which are necessary to align the past image 31 with the imaged image 33 as described above, are calculated.

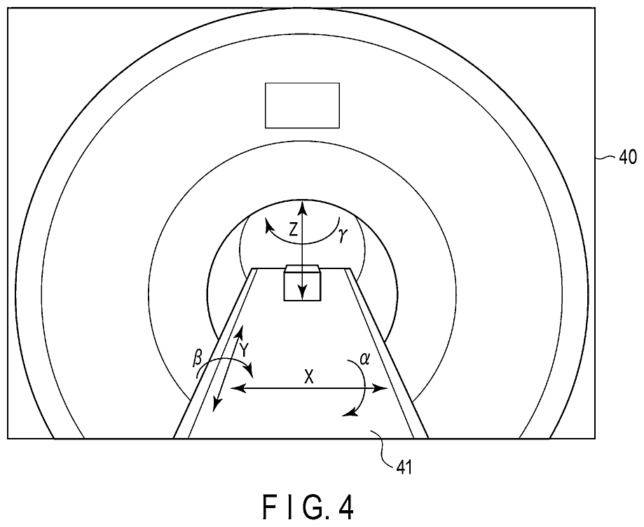

Next, an example of transformation parameters according to the present embodiment will be described with reference to the conceptual diagram of FIG. 4.

FIG. 4 shows an example in which transformation parameters regarding the movement direction and the rotation direction are based on the bed 41 to be inserted into the medical image diagnostic apparatus 40. Herein, the movement direction is defined in three directions, a width direction of the bed 41, a length direction of the bed 41 (the direction in which the bed is inserted into and retreated from the medical image diagnostic apparatus 40), and a height direction perpendicular to the bed 41. The width direction will be referred to as an “X direction”, the length direction will be referred to as a “Y direction”, and the height direction will be referred to as a “Z direction”. Furthermore, the rotation direction is defined in three direction, an α direction around the X axis, a β direction around the Y axis, and a γ direction around the Z axis.

For example, the processing circuitry 10 calculates transformation parameters through the calculation function 102 such that a similarity degree between the imaged image and the past image is maximized. The similarity degree may be calculated using a mean square error, a mutual information, a sum of squared differences, a sum of absolute differences, a normalized cross-correlation, an image uniformity ratio, etc. In a case of linear transformation, for example, a correction value T can be calculated using a transformation expression such as Expression (1) where the aforementioned rotation amounts α, β, and γ, and the movement amount in the X direction, are set to tₓ, the movement amount in the y direction is set to t_{y}, and the movement amount in the z direction is set to t_{z}. Equation (1) is an example, and any method may be adopted as long as an imaged image can be transformed such that the similarity degree between the image and a past image is maximized.

$$T = [ \begin{matrix}cos β cos γ & cos β cos γ + sin α sin β cos γ & sin α sin γ - cos α sin β cos γ & {t}_{x} \\ - cos β sin γ & cos α cos γ - sin α sin β sin γ & sin α cos γ + cos α sin β sin γ & {t}_{y} \\ sin β & - sin α cos β & cos α cos β & {t}_{z} \\ 0 & 0 & 0 & 1\end{matrix} ] \qquad ( 1 )$$

As for how to determine a reference position for transformation (alignment) between the past image and the imaged image, for example, in a case of the medical image diagnostic device being an MRI device or an X-ray CT device, imaging of the past image and the imaged image may be performed while a marker with a contrast agent being encapsulated therein is stuck on a surface of a subject. In a case of the medical image diagnostic apparatus being a PET apparatus or a SPECT apparatus, imaging of the past image and the imaged image may be performed while a marker with a radioactive substance being encapsulated therein is stuck on a surface of a subject.

This requires only linear transformation or nonlinear transformation based on the reference position, thereby making it easier to calculate the correction value. Meanwhile, without being limited to sticking on a marker, linear or nonlinear transformation may be performed based on an anatomical landmark such as a bone or an organ, which is common between the past image and the imaged image. That is, any criterion may be used as long as a correction value can be calculated.

Next, a first application example of a trained model according to the present embodiment will be described with reference to FIG. 5.

In step SA3 shown in FIG. 2, through the model execution function 105, the processing circuitry 10 estimates a transformation parameter 53 by applying a trained model 50 to a past image 51 and an imaged image 52, as shown in FIG. 5. The trained model 50 in this case may be generated by training a machine learning model through supervised learning using data for training in which a past imaged image (also referred to as a “second imaged image”) and a current imaged image (also referred to as a “first imaged image”) both relating to the same imaging target site are set to input data, and a transformation parameter calculated in such a manner as to maximize the similarity degree between the first imaged image and the second imaged image is set to correct answer data. As such a machine learning model, for example, a neural network such as a deep convolutional neural network (DCNN) or bidirectional encoder representations from transformers (BERT) may be used. Since a general training method can be adopted as a training method for the machine learning model, a detailed description of the method is omitted.

Meanwhile, the transformation parameter 53 is not a limitation, and training may be performed such that a correction value 54 is directly output from the trained model 50. That is, a trained model 60 may be generated by training the machine learning model using data for training in which the past image 51 and the imaged image 52 are set to input data, and the correction value obtained based on the transformation parameter based on the past image 51 and the imaged image 52 is set to correct answer data. By this, through the model execution function 105, the processing circuitry 10 can estimate the correction value 54 of the imaged image 52 with respect to the past image 51 by applying the trained model 50 to the past image 51 and the imaged image 52. Therefore, the processing in step SA3 and the processing in step SA4 shown in FIG. 2 can be executed at once using the trained model 60.

Next, a second application example of the trained model according to the embodiment will be described with reference to FIG. 6.

The embodiment described above assumes that the correction value is calculated based on a positioning image or an actual imaged image through the main scan in order to make a comparison with a past image; however, the present invention is not limited to this, and the imaged image may be estimated based on a camera image. The camera image may be acquired by photographing with a camera device installed on, for example, a ceiling of an examination room or upper side of gantry of the medical image diagnostic apparatus. Meanwhile, the camera image may be photographed by a user holding a device equipped with a camera mechanism, such as a tablet terminal, or fixing the device on a tripod, etc. In other words, any photographing means that can photograph a camera image may be used.

As shown in FIG. 6, through the model execution function 105, the processing circuitry 10 generates a cross section image 62 by applying the trained model 60 to the camera image 61. The trained model 60 may be generated by training the machine learning model through supervised learning while a camera image photographed upon completion of a setting of an imaging target site on a subject is set to input data and a cross section image of the imaging target site imaged with the aforementioned setting is set to correct answer data. In addition to the neural network described above, the machine learning model may use a generative neural network such as generative adversarial networks (GAN), conditional GAN (cGAN), conditional variable auto-encoder (cVAE), etc. In a case of using GAN, etc., a trained model trained through so-called semi-supervised learning or unsupervised learning may be generated.

In other words, the trained model has learned what kind of imaged image of a cross section can be obtained based on the site of the subject that is represented in the camera image 61 or the position and the angle of the site. Thus, the cross section image required for image alignment with the past image can be estimated.

By this, in step SA2 shown in FIG. 2, the imaged image can be acquired by photographing a camera image and applying the trained model to the camera image, instead of actual execution of imaging on the same object as that of the past image. By this, the reproducibility of the past image can be achieved with a setting in which, for example, the subject is placed on a bed. Thus, the process of actual photographing of an image can be omitted to improve the efficiency. Furthermore, in a case of a medical image being an X-ray image, a CT image, a PET image, or a SPECT image, exposure to the subject can also be reduced.

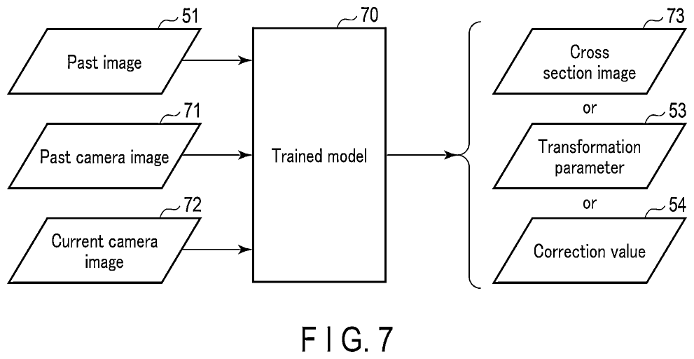

Next, a third application example of the trained model according to the present embodiment will be described with reference to FIG. 7.

As shown in FIG. 7, a cross section image 73 is generated by inputting the past image 51 of the same subject, a camera image with a setting at the time of photographing the past image 51 (hereinafter also referred to as a “past camera image 71” or a “fourth camera image”), and a camera image with a current setting (hereinafter also referred to as a “current camera image 72” or a “third camera image”) to a trained model 70. The trained model 70 in a case of estimating the cross section image 73 is generated by training the machine learning model while a past image, a camera image (also referred to as a “second camera image”) according to a setting before the past image was photographed and a different camera image (also referred to as a “first camera image”) from the second camera image in the same imaging target site are set to input data, and a medical image presenting a cross section photographed with a setting photographed in the aforementioned different camera image is set to correct answer data. As compared to the trained model 50 shown in FIG. 5, it is assumed that the cross section image further reflecting a correspondence relationship between past images and past camera images is generated by using more past images and past camera images for training.

Meanwhile, as with FIG. 5, the processing circuitry 10 may estimate, through the model execution function 105, the transformation parameter 53 or the correction value 54 using the past image 51, the past camera image 71, and the current camera image 72. Specifically, the trained model 70 is generated by training the machine learning model through supervised learning while a past camera image photographed upon completion of a setting of an imaging target site on a subject, a past image imaged with the aforementioned setting, and a camera image according to a setting at the time of photographing a different medical image is set to input data, and transformation parameters relating to the different medical image and the past image are set to correct answer data. This enables estimation of the transformation parameter 53 indicative of a difference between the medical image imaged with the setting of the current camera image 72 and the past image 51.

Similarly, the trained model 70 is obtained by training the machine learning model through supervised learning in which a past camera image photographed upon completion of a setting of an imaging target site on a subject, a past image imaged with the aforementioned setting, and a camera image according to setting at the time of photographing a different medical image are set to input data, and correction values based on transformation parameters relating to the different medical image and the past image are set to correct answer data. This enables estimation of the correction value 54 for reproduction of the past image 51 with respect to a medical image imaged with the setting of the current camera image 72.

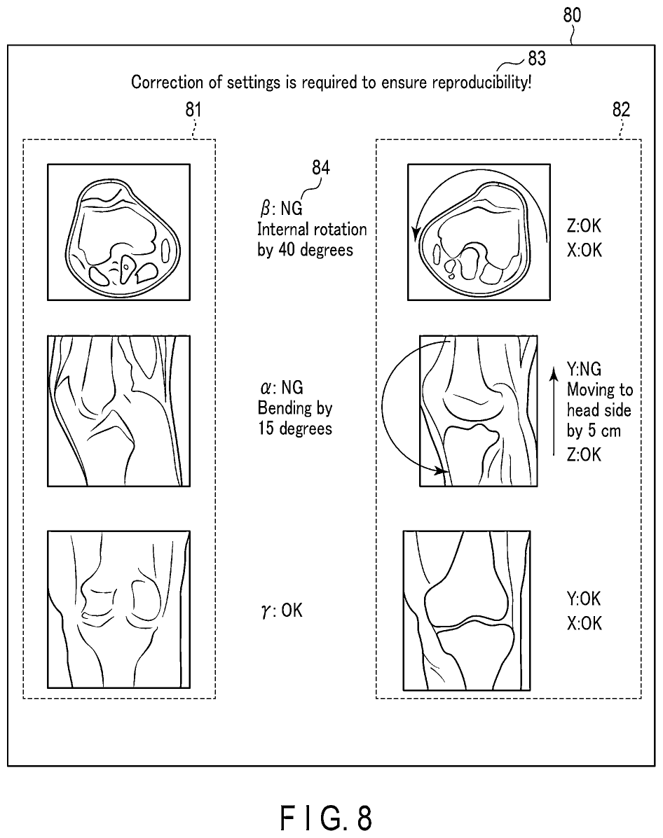

Next, an example of a display screen relating to a correction value will be described with reference to FIG. 8.

FIG. 8 is an example of a user interface for a correction value to be presented to a user. The display screen 80 displays past images 81 showing three different cross sections, and current images 82 showing three cross sections respectively corresponding to the past images 81.

If a user views the display screen 80, he or she can understand the need for correction through a message 83 saying that “correction of settings is required to ensure reproducibility!”. Furthermore, according to a message 84 relating to a correction value, the user can understand that two current images 82 respectively in the ZX plane and the YZ plane require correction of the current setting in order to reproduce past images, while with respect to the current image 82 in the XY plane, a corresponding past image can be reproduced using the current setting.

Specifically, the user can understand that the current image 82 in the ZX plane requires correction of the transformation parameter B, and the angle at which the knee is internally rotated is 40 degrees. Similarly, the user can understand that the current image 82 in the YZ plane requires correction of the transformation parameters x and Y, that the angle at which the knee is bent is 15 degrees, and that the movement in a direction of the head is 5 cm. The user can perform imaging which ensures the reproducibility of a past image by moving the subject by 5 cm in the direction of the head (insertion direction), bending the knees by 15 degrees, and correcting the setting such that the knee is internally rotated by 40 degrees.

The coordinate system of an image (referred to as an “image coordinate system”) is different from the coordinate system relating to positional correction of the subject in the examination room (referred to as a “measurement coordinate system”). Thus, it is necessary to match the coordinate systems. The transformation parameter is calculated in the image coordinate system. Thus, the correction value is calculated by, for example, executing transformation processing to the measurement coordinate system on the conversion parameter in the image coordinate system. In the transformation processing, for example, the transformation parameter is multiplied by a parameter for alignment of the coordinates acquired from data such as header information of an image. By this, coordinate alignment is performed to calculate a correction value. This is not a limitation, and any processing may be used as long as the image coordinate system can be transformed to the measurement coordinate system.

The instruction function 104 may also determine the order in which a subject is rotated or moved, depending on the type of transformation parameter and the correction value. For example, in a case of correction of the setting in the YZ plane shown in FIG. 8, it is assumed that a shift occurs in the X direction or the Y direction as the knee is bent. Thus, if correction relating to the transformation parameter X or Y relating to the movement amount is made in advance, a difference may occur in correction of the conversion parameter a. For this reason, correction may be made in the order in which correction is made, first to the transformation parameter a, that is, the bending angle of the knee, and then to the movement Y.

For such an order of correction, for example, a table is prepared in advance that defines the order of correction according to, for example, the imaging target site, the type of transformation parameter that requires correction, and the range of correction values. The processing circuitry 10 may determine, through the instruction function 104, the order of correction of the rotation and movement based on transformation parameters by referring to the transformation parameters calculated through the calculation function 102 and the aforementioned table. Alternatively, the trained model may be generated by training a machine learning model based on a relationship between the order of correction and both the transformation parameters and correction values, which have already been implemented. By this, the order of correction can be estimated by inputting the transformation parameters and correction values obtained through the calculation function 102 into the trained model. Meanwhile, the machine learning model may be trained such that the order of correction is also output as output of the trained model shown in FIG. 5 or FIG. 7.

Furthermore, through the display control function 106, the processing circuitry 10 may display the transformation parameters and the correction values in the order of correction from the top to the bottom of the display screen 90. This allows a user to efficiently correct a setting.

The present embodiment described above acquires, with respect to the same subject, a current imaged image or camera image and a past image imaged in the past, calculates a correction value for reproducing the past image, and determine, based on the correction value, reproducibility regarding whether or not the past image is reproducible. In a case where a correction value falls within a range that allows reproduction of past images, the setting of the subject may be corrected based on the correction value. This can improve the reproducibility of the imaging cross section. This enables an image with high reproducibility of the cross section to be collected regardless of the operator during, for example, follow-up observation. Thus, the quality and speed of image diagnosis can be improved.

Various functions of the processing circuitry 10 may be stored in the memory 11 in the form of a program executable by a computer. In this case, the processing circuitry 10 can also be regarded as a processor which reads programs corresponding to these various functions from the memory 11 and executes the read programs to realize functions corresponding to the respective programs. In other words, the processing circuitry 10 that has read the programs has, for example, the functions shown in the processing circuitry 10 shown in FIG. 1.

FIG. 1 illustrates the case in which various functions are realized in the single processing circuitry 10; however, the processing circuitry 10 may be constituted by a combination of a plurality of independent processors, and the functions may be realized by the processors executing the programs. In other words, each of the above-described functions may be formed as a program, and a single processing circuitry may execute each program, or a specific function may be implemented in dedicated, independent program-execution circuitry.

According to at least one of the embodiments described above, reproducibility of an imaging cross section can be improved.

Moreover, the functions described in connection with the above embodiment may be implemented, for example, by installing a program for executing the processing in a computer, such as a workstation, etc., and expanding the program in a memory. The program that causes the computer to execute the processing can be stored and distributed by means of a storage medium, such as a magnetic disk (a hard disk, etc.), an optical disk (CD-ROM, DVD, etc.), and a semiconductor memory.

Herein, the term “processor” used in the above explanation means, for example, a circuitry such as a central processing unit (CPU), a graphics processing unit (GPU), an application-specific integrated circuitry (ASIC), or a programmable logic device (e.g., a simple programmable logic device (SPLD), a complex programmable logic device (CPLD), or a field-programmable gate array (FPGA)).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An imaging support apparatus comprising processing circuitry configured to:
acquire a first image and a second image older than the first image;
calculate a correction value for reproducing the second image; and
determine reproducibility of the second image based on the correction value.

2. The imaging support apparatus according to claim 1, wherein the reproducibility represents a criterion to determine whether or not a setting for imaging based on the correction value is possible for an imaging target site.

3. The imaging support apparatus according to claim 2, wherein the processing circuitry is further configured to output a correction instruction for the setting in a case where the setting based on the correction value is possible for the imaging target site.

4. The imaging support apparatus according to claim 2, wherein the processing circuitry is further configured to output information of an unreproducible status in a case where the setting based on the correction value is impossible for the imaging target site.

5. The imaging support apparatus according to claim 2, wherein the processing circuitry is further configured to present a correction instruction of the setting for making an image closest to the second image and a difference from the second image as corrected based on the correction instruction in a case where the setting based on the correction value is impossible for the imaging target site.

6. The imaging support apparatus according to claim 3, wherein the processing circuitry is configured to execute at least one of presentation of the correction value to an operator or a control instruction for moving or rotating the imaging target site by driving a device.

7. The imaging support apparatus according to claim 1, wherein the processing circuitry is configured to calculate a transformation parameter such that a similarity degree to the second image is maximized through transformation processing on the first image, and to calculate the correction value from the transformation parameter.

8. The imaging support apparatus according to claim 7, wherein the similarity degree is a value of at least one of a mean square error, a mutual information, a sum of squared differences, a sum of absolute differences, a normalized cross-correlation, or an image uniformity ratio.

9. The imaging support apparatus according to claim 7, wherein the correction value is a movement amount and a rotation angle of an imaging target site extracted in the first image, the movement amount and the rotation angle being calculated from the transformation parameter.

10. The imaging support apparatus according to claim 1, wherein the processing circuitry is further configured to generate a transformation parameter by inputting the first image and the second image to a trained model, and generate the correction value from the transformation parameter inferred by the trained model, the trained model being trained while a first imaged image and a second imaged image of an imaging target site are set to input data, the second imaged image being imaged before the first imaged image, and the transformation parameter calculated such that a similarity degree to the second imaged image is maximized through transformation processing on the first imaged image is set to correct answer data.

11. The imaging support apparatus according to claim 1, wherein the processing circuitry is further configured to generate, as the first image, a cross section image relating to an imaging target site by inputting a camera image obtained by photographing the imaging target site with a camera device to a trained model, the trained model being trained while a camera image obtained by photographing the imaging target site with the camera device is set to input data and a cross section image of the imaging target site imaged with a medical image diagnostic apparatus is set to correct answer data.

12. The imaging support apparatus according to claim 1, wherein the processing circuitry is further configured to generate a transformation parameter by inputting a third camera image obtained by photographing an imaging target site with a camera device, the second image, and a fourth camera image corresponding to the second image to a trained model, and generate the correction value from the transformation parameter inferred by the trained model, the trained model being trained while a first camera image obtained by photographing the imaging target site with the camera device, a second camera image different from the first camera image and relating to the imaging target site, and a past imaged image obtained by imaging the imaging target site with a medical image diagnostic apparatus are set to input data and the transformation parameter calculated such that a similarity degree to the past imaged image is maximized in a case in which an imaged image corresponding to the first camera image is assumed is set to correct answer data.

13. The imaging support apparatus according to claim 1, wherein the first image is a medical image collected from a same imaging target site relating to the second image using a medical image diagnostic apparatus or a camera device.

14. The imaging support apparatus according to claim 1, wherein the first image and the second image are medical images collected from a same imaging target site using a same medical image diagnostic apparatus or different medical image diagnostic apparatuses.

15. An imaging support method comprising:
acquiring a first image and a second image older than the first image;
calculating a correction value for reproducing the second image; and
determining reproducibility of the second image based on the correction value.

16. A non-transitory computer readable medium including computer executable instructions, wherein the instructions, when executed by a processor, cause the processor to perform a method comprising:
acquiring a first image and a second image older than the first image;
calculating a correction value for reproducing the second image; and
determining reproducibility of the second image based on the correction value.
